(19) United States
(12) Patent Application Publication
(10) Pub. No.: US 2024/0041125 A1
(43) Pub. Date: Feb. 8, 2024

(54) VAPORIZER AND ELECTRONIC VAPORIZATION DEVICE

(71) Applicant: Shenzhen Verdewell Technology Limited, Shenzhen (CN)

(72) Inventor: Ju XIE, Shenzhen (CN)

(21) Appl. No.: 18/358,979

(22) Filed: Jul. 26, 2023

(30) Foreign Application Priority Data
Aug. 4, 2022 (CN) 202222048048.2

(51) Int. Cl. A24F 40/48; A24F 40/10; A24F 40/44; A24F 40/42; A61M 11/04

(52) U.S. Cl. CPC A24F 40/48; A24F 40/10; A24F 40/44; A24F 40/42; A61M 11/042

(57) ABSTRACT

A vaporizer includes: a tube body having a liquid guide channel therein; a liquid guide member having a liquid guide surface and a vaporization surface arranged opposite each other, the liquid guide surface being in communication with a liquid inlet end of the liquid guide channel so as to guide an aerosol-forming substrate from the liquid guide surface to the vaporization surface; and a heating element arranged on the vaporization surface for heating and vaporizing the aerosol-forming substrate. The tube body includes an air inlet channel. The vaporization surface is in communication with the air inlet channel. An airflow channel is constructed inside the tube body. Two opposite ends of the airflow channel are respectively in communication with the liquid inlet end of the liquid guide channel and the vaporization surface.

### CROSS-REFERENCE TO PRIOR APPLICATION

Priority is claimed to Chinese Patent Application No. 202222048048.2, filed on Aug. 4, 2022, the entire disclosure of which is hereby incorporated by reference herein.

### FIELD

This application relates to the technical field of vaporizers, and in particular, to a vaporizer and an electronic vaporization device.

### BACKGROUND

An electronic vaporization device is a device that uses a liquid guide member for absorption and guides an aerosol-forming substrate stored in a liquid storage cavity and then heats the aerosol-forming substrate through a heating element to produce aerosols.

The electronic vaporization device is usually provided with a liquid guide channel to guide the aerosol-forming substrate to the liquid guide member for subsequent vaporization. However, after a period of continuous liquid feeding in the liquid guide channel, negative pressure is generated at a liquid inlet end of the liquid guide channel, which affects the liquid feeding. In order to avoid the case, the electronic vaporization device is further provided with a vent hole or a vent slot to replenish pressure for the liquid inlet end of the liquid guide channel, thereby ensuring air replenishment for the air inlet end of the liquid guide channel.

However, due to factors such as gravity or air pressure, the liquid inside the liquid guide channel seeps back into the vent hole or the vent slot, resulting in leakage during ventilation of the electronic vaporization device. As a result, the use experience of consumers is poor.

### SUMMARY

In an embodiment, the present invention provides a vaporizer, comprising: a tube body having a liquid guide channel therein; a liquid guide member having a liquid guide surface and a vaporization surface arranged opposite each other, the liquid guide surface being in communication with a liquid inlet end of the liquid guide channel and configured to guide an aerosol-forming substrate from the liquid guide surface to the vaporization surface; and a heating element arranged on the vaporization surface and configured to heat and vaporize the aerosol-forming substrate, wherein the tube body comprises an air inlet channel, wherein the vaporization surface is in communication with the air inlet channel, wherein an airflow channel is constructed inside the tube body, and wherein two opposite ends of the airflow channel are respectively in communication with the liquid inlet end of the liquid guide channel and the vaporization surface.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a three-dimensional schematic structural diagram of a vaporizer according to some embodiments of this application.

FIG. 2 is a schematic structural exploded view of the vaporizer according to some embodiments of this application.

FIG. 3 is a partial schematic structural diagram of the vaporizer according to some embodiments of this application.

FIG. 4 is a schematic structural cross-sectional view of the vaporizer according to some embodiments of this application.

FIG. 5 is a partial three-dimensional schematic structural diagram of the vaporizer according to some embodiments of this application.

FIG. 6 is a partial schematic structural cross-sectional view of the vaporizer according to some embodiments of this application.

FIG. 7 is a partial three-dimensional schematic structural diagram of the vaporizer according to some embodiments of this application.

FIG. 8 is a partial three-dimensional schematic structural diagram of the vaporizer according to some embodiments of this application.

### DETAILED DESCRIPTION

In an embodiment, the present invention provides a vaporizer and an electronic vaporization device to address a phenomenon of liquid leakage from a vent position in existing electronic vaporization devices.

In an embodiment, the present invention provides a vaporizer, including:
a tube body, having a liquid guide channel therein;
a liquid guide member, having a liquid guide surface and a vaporization surface arranged opposite to each other, where the liquid guide surface is in communication with a liquid inlet end of the liquid guide channel, and is configured to guide an aerosol-forming substrate from the liquid guide surface to the vaporization surface; and
a heating element, arranged on the vaporization surface and configured to heat and vaporize the aerosol-forming substrate.

The tube body further has an air inlet channel, the vaporization surface is in communication with the air inlet channel, an airflow channel is further constructed inside the tube body, and two opposite ends of the airflow channel are respectively in communication with the liquid inlet end of the liquid guide channel and the vaporization surface.

In an embodiment, the vaporizer includes a vent tube. The vent tube is arranged in the tube body, and the airflow channel is formed inside the vent tube.

In an embodiment, in a direction from the vaporization surface to the liquid guide surface, an end of the airflow channel is flush with the liquid inlet end of the liquid guide channel or the end of the airflow channel is higher than the liquid inlet end of the liquid guide channel.

In an embodiment, the vaporizer further includes a first seal member arranged between the tube body and the liquid guide member.

The liquid guide channel and the airflow channel are both arranged on the first seal member and extend in an axial direction of the tube body.

In an embodiment, the vaporizer further includes an air guide member, the air guide member is arranged in the tube body and located on a side of the liquid guide member facing away from the first seal member, the air guide member, the liquid guide member, and the tube body jointly define a vaporization cavity.

The vaporization surface is arranged facing the vaporization cavity, and the air inlet channel is in communication with the vaporization cavity.

In an embodiment, the air guide member has a first end surface, a vaporization groove with an opening is arranged on the first end surface, and the first end surface is attached to the first seal member for the liquid guide member to cover the opening of the vaporization groove and define the vaporization cavity.

In an embodiment, the air guide member has a first end surface, the first end surface is spaced apart from the liquid guide member in the axial direction of the tube body, and the first end surface, the tube body, and the liquid guide member define the vaporization cavity.

In an embodiment, the air guide member further has a second end surface, the second end surface is in communication with an end of the air inlet channel.

The air guide member has an air guide channel, and two opposite ends of the air guide channel are respectively in communication with the first end surface and the second end surface.

In an embodiment, the vaporizer further includes a housing, the housing is sleeved on an outer periphery of the tube body and defines a liquid storage cavity with the tube body.

The tube body has a liquid inlet hole facing the liquid storage cavity, the liquid inlet hole forms the liquid inlet end of the liquid guide channel, and the liquid storage cavity is in communication with the liquid guide surface through the liquid inlet hole.

In an embodiment, the vent tube includes at least one capillary tube, and a pore size of the capillary tube is in a range of 0.15 mm to 0.45 mm.

According to another aspect of this application, an electronic vaporization device is provided, including the vaporizer in any one of the above embodiments.

In the above vaporizer, the airflow channel is arranged to be in communication with the liquid inlet end of the liquid guide channel and the vaporization surface, the vaporization surface is in communication with the air inlet channel, the air inlet channel is in communication with the liquid inlet end of the liquid guide channel, and the end of the airflow channel in communication with the vaporization surface is close to the heating element. During heating of the vaporization surface by the heating element, the heat of the heating element is transmitted to the aerosol-forming substrate near the liquid inlet end of the liquid guide channel through the airflow channel, to reduce the viscosity of the aerosol-forming substrate, so as to reduce the resistance to air replenishment from the airflow channel to the liquid inlet end, thereby facilitating successful entry of a venting airflow in the airflow channel to the liquid inlet end for air replenishment. Moreover, the liquid is not prone to reverse leakage from the airflow channel during high-speed ventilation in the airflow channel.

Reference Numerals: 100. Vaporizer; 10. Housing; 11. Mouthpiece; 12. Shell; 20. Tube body; L: Axial direction; 21. Upper tube body; 211. Central flow channel; 212. Liquid inlet hole; 22. Fastening ring; 23. Heating base; 231. Negative connecting member; 232. Air inlet channel; Liquid guide member; 31. Liquid guide surface; 32. Vaporization surface; 50. First seal member; 51. Liquid guide channel; 51a. Liquid inlet end; 51b. Liquid outlet end; 52. First mounting hole; 60. Vent tube; 61. Airflow channel; 70. Air guide member; 71. First end surface; 711. Vaporization groove; 712. Avoidance position; 72. Second end surface; 73: Air guide channel; 74. Second mounting hole; a. Liquid storage cavity; b: Vaporization cavity; 80. Second seal member; 90. Positive connecting member.

In order to make the foregoing objectives, features, and advantages of this application clearer and easier to understand, specific implementations of this application are described in detail below with reference to the drawings. In the following description, many specific details are described for thorough understanding of this application. However, this application may be implemented in many other manners different from those described herein. A person skilled in the art may make similar improvements without departing from the connotation of this application. Therefore, this application is not limited to the specific embodiments disclosed below.

In the description of this application, it should be understood that orientation or position relationships indicated by the terms such as “center”, “longitudinal”, “transverse”, “length”, “width”, “thickness”, “up”, “down”, “front”, “back”, “left”, “right”, “vertical”, “horizontal”, “top”, “bottom”, “inside”, “outside”, “clockwise”, “anticlockwise”, “axial direction”, “radial direction”, and “circumferential direction” are based on orientation or position relationships shown in the drawings, and are used only for ease and brevity of description of this application, rather than indicating or implying that the mentioned device or element needs have a particular orientation or be constructed and operated in a particular orientation. Therefore, such terms should not be construed as a limitation on this application.

Furthermore, the terms “first” and “second” are merely used for descriptive purpose, and should not be interpreted as indicating or implying relative significance or implicitly indicating a number of the indicated technical features. Therefore, the features defined with “first” and “second” may include at least one of the features explicitly or implicitly. In the description of this application, “a plurality of” means at least two, such as two or three, unless otherwise definitely and specifically defined.

In this application, unless otherwise explicitly specified or defined, the terms such as “mount”, “connect”, “connection”, and “fix” should be understood in a broad sense. For example, the connection may be a fixed connection, a detachable connection, or an integral connection; or may be a mechanical connection or an electrical connection; or may be a direct connection, an indirect connection through an intermediate medium, internal communication between two elements, or an interaction relationship between two elements, unless otherwise definitely defined. A person of ordinary skill in the art may understand the specific meanings of the above terms in the present disclosure according to specific situations.

In this application, unless otherwise explicitly specified and defined, a first feature being “on” or “under” a second feature may mean that the first feature is in direct contact with the second feature, or the first feature is in indirect contact with the second feature by using an intermediate medium. Moreover, the first feature being “over”, “above”, and “on” the second feature may mean that the first feature is directly above or obliquely above the second feature, or merely means that the first feature has a larger horizontal height than the second feature. The first feature being “under”, “below” and “underneath” the second feature may mean that the first feature is directly below or obliquely below the second feature, or merely means that the first feature has a smaller horizontal height than the second feature.

It should be noted that, when an element is referred to as “being fixed to” or “being arranged on” another element, the element may be directly located on the another element, or an intermediate element may exist. When an element is considered to be “connected to” another element, the element may be directly connected to the another element, or an intermediate element may exist. The terms “vertical”, “horizontal”, “upper”, “lower”, “left”, “right”, and similar expressions used herein are used for illustration but do not indicate a unique implementation.

As described in the background, electronic vaporization devices become increasingly popular among consumers in recent years. Current electronic vaporization devices in the market generally include a vaporizer and electrical elements. A vaporization assembly is configured to vaporize a volatile aerosol-forming substrate into an inhalable aerosol. During continuous liquid feeding and vaporization in a liquid guide channel, negative pressure is gradually generated at a liquid inlet end of the liquid guide channel, which affects a liquid supply speed, resulting in “unsmooth liquid feeding”.

In order to avoid the case, the electronic vaporization devices are usually provided with a vent hole or vent slot, which is in direct communication with the external air to directly replenish the external air through the liquid inlet end of the liquid guide channel, thereby preventing liquid feeding from being affected by the negative pressure at the liquid inlet end of the liquid guide channel.

However, due to factors such as gravity or air pressure, during air replenishment for the liquid inlet section of the liquid guide channel, the liquid inside the liquid guide channel seeps back into the vent hole or the vent slot, resulting in leakage during ventilation of the electronic vaporization device. As a result, the use experience of consumers is poor.

In order to resolve the above problem, this application provides an electronic vaporization device. The electronic vaporization device may be configured to vaporize liquid media such as smoke liquid and drugs. The electronic vaporization device may include a vaporizer and electrical elements electrically connected to the vaporizer to supply power to the vaporizer. The vaporizer is configured to heat and vaporize a liquid aerosol-forming substrate, and the electrical elements are configured to supply power to the vaporizer.

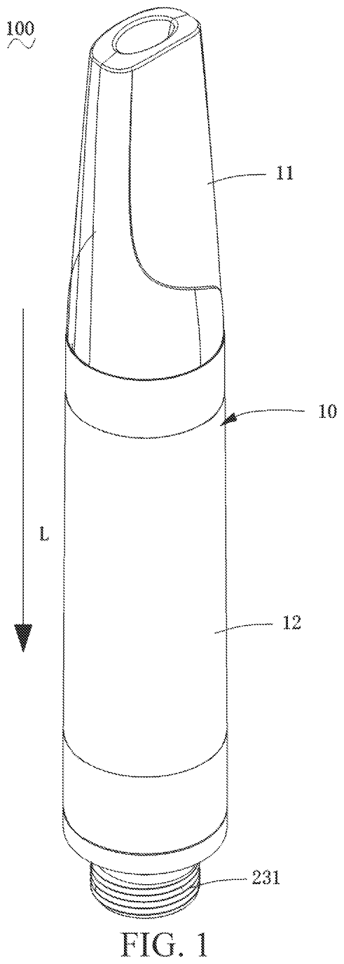

Referring to FIG. 1 to FIG. 3, an embodiment of this application provides a vaporizer 100 for an electronic vaporization device, including a tube body 20, a liquid guide member 30, and a heating element. The tube body 20 has a liquid guide channel 51 therein. It may be understood that the liquid guide channel 51 has a liquid inlet end 51a and a liquid outlet end 51b. The liquid guide channel 51 is configured to guide an aerosol-forming substrate from the liquid inlet end 51a to the liquid outlet end 51b. The liquid guide member 30 has a liquid guide surface 31 and a vaporization surface 32 arranged opposite to each other. The liquid guide surface 31 is in communication with the liquid inlet end 51a of the liquid guide channel 51, and is configured to guide the aerosol-forming substrate from the liquid guide surface 31 to the vaporization surface 32. The heating element is arranged on the vaporization surface 32 and is configured to heat and vaporize the aerosol-forming substrate.

The tube body 20 further has an air inlet channel 232. The vaporization surface 32 is in communication with the air inlet channel 232. The other end of the air inlet channel 232 is in communication with the external atmosphere. An airflow channel 61 is further constructed inside the tube body 20. Two opposite ends of the airflow channel 61 are respectively in communication with the liquid inlet end 51a of the liquid guide channel 51 and the vaporization surface 32.

The airflow channel 61 is a channel for airflow circulation. The airflow channel 61 is arranged to be in communication with the liquid inlet end 51a of the liquid guide channel 51 and the vaporization surface 32, the vaporization surface 32 is in communication with the air inlet channel 232, the air inlet channel 232 is in communication with the liquid inlet end 51a of the liquid guide channel 51, and the end of the airflow channel 61 in communication with the vaporization surface 32 is close to the heating element. During heating of the vaporization surface 32 by the heating element, the heat of the heating element is transmitted to the aerosol-forming substrate near the liquid inlet end 51a of the liquid guide channel 51 through the airflow channel 61, to reduce the viscosity of the aerosol-forming substrate, so as to reduce the resistance to air replenishment from the airflow channel 61 to the liquid inlet end 51a, thereby facilitating successful entry of a venting airflow in the airflow channel 61 to the liquid inlet end 51a for air replenishment.

Moreover, while the airflow is rapidly replenished through the airflow channel 61, the airflow continuously flows in the airflow channel 61. The presence of the airflow can prevent reverse leakage of liquid through the airflow channel 61, thereby ensuring smooth ventilation while avoiding reverse leakage.

Specifically, the aerosol-forming substrate introduced through the liquid inlet end 51a of the liquid guide channel 51 may be stored in the vaporizer 100 or provided through external devices. This is not limited herein in this application.

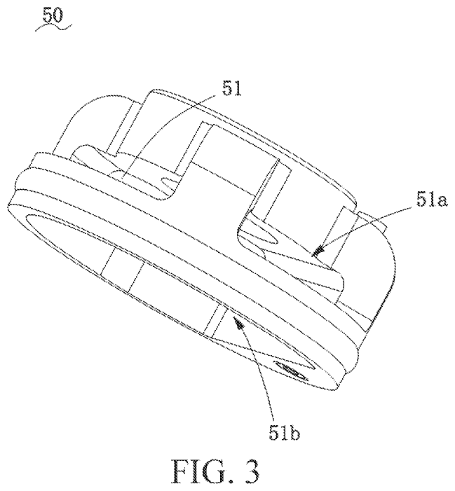

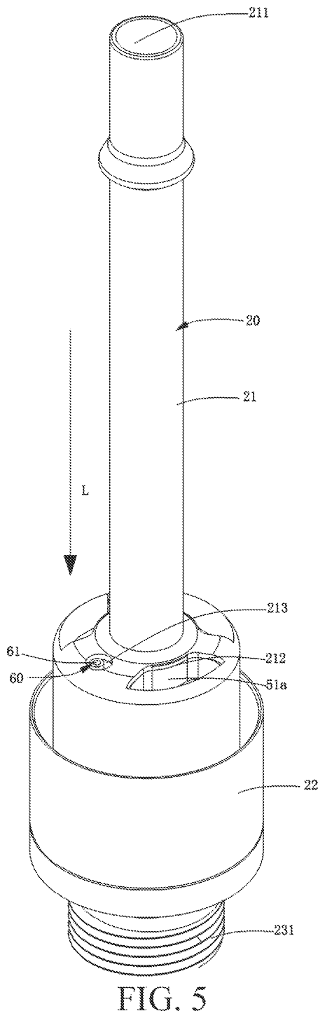

In an embodiment, referring to FIG. 3 and FIG. 5, the vaporizer 100 further includes a housing 10. The housing 10 is sleeved around an outer periphery of the tube body 20 and defines a liquid storage cavity a with the tube body 20. The tube body 20 has a liquid inlet hole 212 facing the liquid storage cavity a, the liquid inlet hole 212 forms the liquid inlet end 51a of the liquid guide channel 51, and the liquid storage cavity a is in communication with the liquid guide surface 31 through the liquid inlet hole 212.

When a user needs to use the electronic vaporization device, the aerosol-forming substrate stored in the liquid storage cavity a enters the liquid guide channel 51 through the liquid inlet hole 212, and then flows to the liquid guide surface 31 and flows to the vaporization surface 32 through the liquid guide effect of the liquid guide member 30, and is heated and vaporized by the heating element, thereby generating an aerosol.

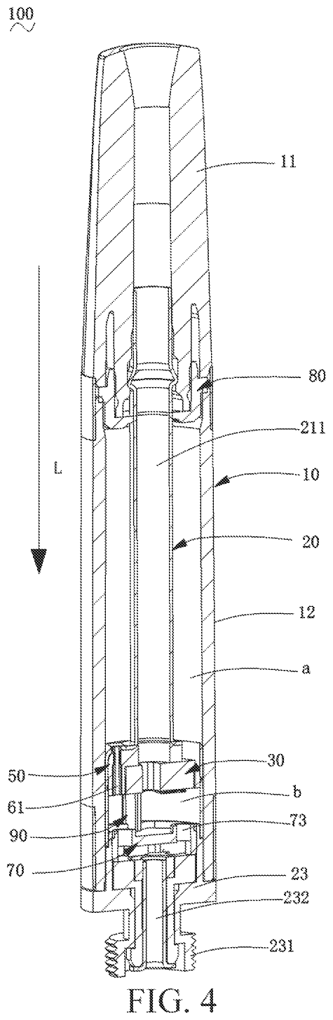

Specifically, referring to FIG. 3 to FIG. 5, the housing 10 includes a mouthpiece 11 and a shell 12 connected to each other, the tube body 20 includes an upper tube body 21, a heating base 23, and a fastening ring 22 connected to each other, the liquid guide member 30 and the heating element are assembled in the upper tube body 21, the heating base 23 is connected to an end in an axial direction L of the upper tube body 21, the air inlet channel 232 is arranged in the heating base 23, the fastening ring 22 is firmly sleeved outside the upper tube body 21 and the heating base 23, a central flow channel 211 is formed inside the upper tube body 21, and two opposite ends of the central flow channel 211 are in communication with the mouthpiece 11 and the air inlet channel 232. The aerosol generated through vaporization on the side of the vaporization surface 32 is carried away by an airflow entering the air inlet channel 232, and is guided by the central flow channel 211 to enter a mouth of the user through the mouthpiece 11 with inhalation of the user.

In an embodiment, the mouthpiece 11, the shell 12, the upper tube body 21, and the heating base 23 jointly define the liquid storage cavity a. The vaporizer 100 further includes a second seal member 80. The second seal member 80 is located at a connection between the mouthpiece 11 and shell 12 and is sleeved around an outer periphery of the upper tube body 21 to seal the liquid storage cavity a.

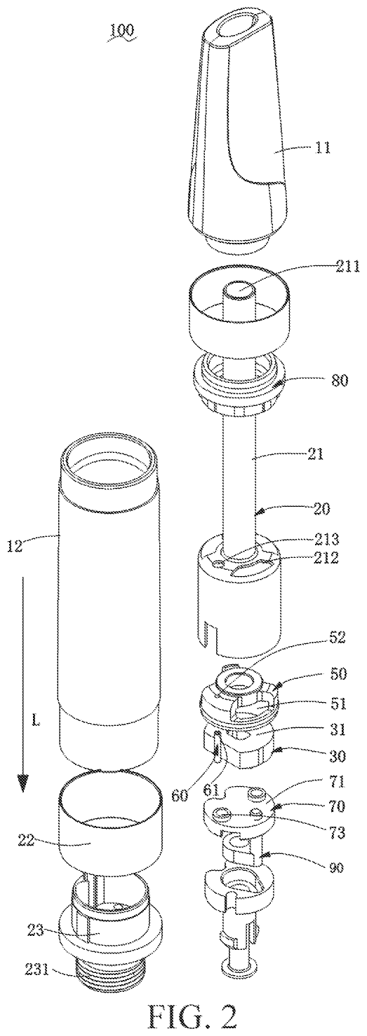

In an embodiment, referring to FIG. 2, the vaporizer 100 includes a vent tube 60. The vent tube 60 is arranged in the tube body 20, and the airflow channel 61 is formed inside the vent tube.

The vent tube 60 is mounted inside the tube body 20 as close as possible to the heating element. The heat from the heating element is transmitted to the aerosol-forming substrate of the vent tube 60 close to the air inlet end of the liquid guide channel 51. The viscosity of the aerosol-forming substrate rapidly decreases when heated, which facilitates separation and rise of external air from the vent tube 60 and increases the ventilation rate.

In other embodiments, the airflow channel 61 may alternatively be constructed on the structure inside the housing 10. In this case, an additional vent tube 60 is not required, which reduces a number of components in the vaporizer 100, thereby simplifying the assembly.

Preferably, the vent tube 60 may be a tube with a very small inner diameter, which can provide a directional capillary effect, to guide the external air entering the air inlet channel 232 to flow to the liquid inlet end 51a of the liquid guide channel 51 through the vent tube 60, and prevent leakage as a result of the liquid flowing back from the vent tube 60. Moreover, due to a small circulation cross-sectional area of the vent tube 60, liquid leakage can be further prevented.

In an embodiment, the vent tube 60 includes at least one capillary tube. The capillary tube may be made of metal or plastic. The airflow channel 61 in each capillary tube is a microchannel that extends in a cylindrical shape, and has a pore size in a range of 0.15 mm to 0.45 mm, which is preferably 0.3 mm, to ensure a ventilation effect while preventing leakage.

In an embodiment, in a direction from the vaporization surface 32 to the liquid guide surface 31, an end of the airflow channel 61 is flush with the liquid inlet end 51a of the liquid guide channel 51 or the end of the airflow channel 61 is higher than the liquid inlet end 51a of the liquid guide channel 51.

The end of the airflow channel 61 in communication with the liquid inlet end 51a of the liquid guide channel 51 is an air outlet end. External air entering the housing 10 through the air inlet channel 232 enters through an end of the airflow channel 61 and flows out through the air outlet end of the airflow channel 61.

Since the end of the airflow channel 61 is constructed to be flush with the liquid inlet end 51a of the liquid guide channel 51 or the end of the airflow channel 61 is constructed to be higher than the liquid inlet end 51a of the liquid guide channel 51, the air outlet end of the airflow channel 61 is higher than or flush with the liquid inlet end 51a of the liquid guide channel 51, which prevents the aerosol-forming substrate at the liquid inlet end 51a from blocking the air outlet end of the airflow channel 61 and causing blockage or bubble jamming in the airflow channel 61, thereby ensuring smooth airflow.

In an embodiment, referring to FIG. 2 to FIG. 4, the vaporizer 100 further includes a first seal member 50. The first seal member 50 is arranged between the tube body 20 and the liquid guide member 30 to ensure the sealing between the liquid guide member 30 and the housing 10. In this case, the aerosol-forming substrate introduced through the liquid inlet end 51a of the liquid guide channel 51 will not leak from a space between the tube body 20 and the liquid guide member thereby ensuring the liquid supply rate.

Further, the liquid guide channel 51 and the airflow channel 61 are both arranged on the first seal member 50 and extend in the axial direction L of the tube body 20. The liquid guide channel 51 may be formed by forming holes and grooves on the first seal member 50, and the airflow channel 61 may be directly constructed on the first seal member 50. Alternatively, as shown in FIG. 2, a first mounting hole 52 may be arranged, and the vent tube 60 in the above is assembled on the first seal member 50, to form the airflow channel.

In an embodiment, when the first mounting hole 52 is arranged on the first seal member the vent tube 60 is assembled in the first mounting hole 52 to form the airflow channel 61. In this case, the first mounting hole 52 may form a stepped mounting hole along an extending direction thereof to achieve an interference fit with the vent tube 60 at a position of the first mounting hole 52 with a small pore diameter, thereby preventing the aerosol-forming substrate in the liquid storage cavity a from leaking from the first mounting hole 52.

The liquid guide channel 51 and the airflow channel 61 both extend in the axial direction L of the tube body 20, to keep the liquid inlet end 51a of the liquid guide channel 51 and the air outlet end of the airflow channel 61 at the same position, that is, both are in communication with the liquid storage cavity a, and to keep the liquid outlet end 51b of the liquid guide channel 51 and the air inlet end of the airflow channel 61 at the same position, that is, both are in communication with the liquid guide member 30, so that external air can be replenished through the airflow channel 61. The aerosol-forming substrate in the liquid storage cavity a supplies liquid to the liquid guide member 30 through the liquid guide channel 51. In this case, a liquid supply direction in the liquid guide channel 51 is opposite to an airflow direction in the airflow channel 61.

Moreover, the airflow channel 61 extends in the axial direction L of the tube body 20 to a position close to the heating element, so that the heat of the heating element is transmitted to the aerosol-forming substrate of the liquid storage cavity a near the liquid inlet end 51a of the liquid guide channel 51 through the airflow channel 61, thereby reducing the viscosity of the aerosol-forming substrate, and thus reducing the resistance to air replenishment through the airflow channel 61.

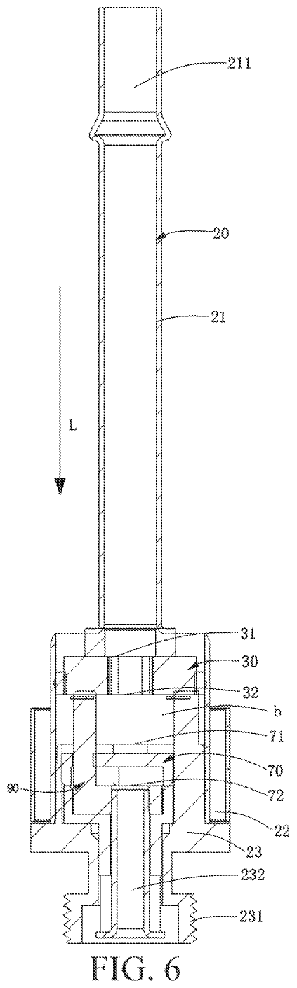

In an embodiment, referring to FIG. 2 and FIG. 6, the vaporizer 100 further includes an air guide member 70. The air guide member 70 is arranged in the tube body 20 and located on a side of the liquid guide member 30 facing away from the first seal member 50. The air guide member 70, the liquid guide member 30, and the tube body 20 jointly define a vaporization cavity b. The vaporization surface 32 is arranged facing the vaporization cavity b, so that the aerosol-forming substrate on the side of the vaporization surface 32 directly enters the vaporization cavity b.

Moreover, the air inlet channel 232 is in communication with the vaporization cavity b, and the other end of the vaporization cavity b is in communication with the central flow channel 211. In this case, the external air entering the air inlet channel 232 has two functions: carrying away the aerosol inside the vaporization cavity b and flowing into the central flow channel 211, and entering the liquid inlet end 51a of the liquid guide channel 51 and the liquid storage cavity a through the airflow channel 61 for air replenishment.

In an embodiment, the air guide member 70 has an air guide channel 73 extending through the axial direction L of the tube body 20, and two opposite ends of the air guide channel 73 are respectively in communication with the air inlet channel 232, to achieve the air guide function.

Further, in an embodiment, referring to FIG. 2 and FIG. 6, the air guide member 70 has a first end surface 71. The first end surface 71 is spaced apart from the liquid guide member 30 in the axial direction L of the tube body 20, and the first end surface 71, the tube body 20, and the liquid guide member 30 define the vaporization cavity b. In this case, a specific spacing exists between the air guide member 70 and the liquid guide member 30 and the first seal member 5050. Air flowing from the air guide channel 73 to the first end surface 71 is shunted in the vaporization cavity b for further flowing.

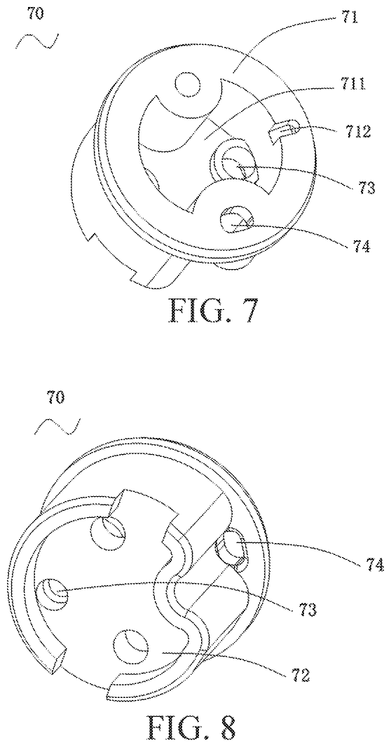

Further, in other embodiments, referring to FIG. 7 and FIG. 8, a vaporization groove 711 with an opening is arranged on the first end surface 71, and the first end surface 71 is attached to the first seal member 50 for the liquid guide member 30 to cover the opening of the vaporization groove 711 and define the vaporization cavity b. In this case, no spacing exists between the air guide member 70 and the liquid guide member 30 and the first seal member 50, and the thickness of the air guide member 70 is increased, so that air flowing from the air guide channel 73 to the first end surface 71 directly enters the vaporization cavity b.

In this way, the thickness of the air guide member 70 is increased, so that the air guide member 70 can abut with the first seal member 50 to achieve double sealing, thereby preventing liquid leakage between the liquid guide member 30 and the tube body 20, and thus preventing air leakage between the liquid guide member 30 and the tube body 20.

Moreover, in this case, the air guide channel 73 may be arranged close to the air inlet channel 232, to reduce the air flow path and flow losses, and ensure the air inlet rate and vaporization rate.

Specifically, referring to FIG. 7, the first end surface 71 further has an avoidance position 712. The avoidance position 712 is in communication with the vaporization cavity b and the airflow channel 61. A part of the air entering the vaporization cavity b enters the airflow channel 61 through the avoidance position 712, and the other part directly impacts the vaporization surface 32 to carry away the aerosol.

In an embodiment, referring to FIG. 6, the air guide member 70 further has a second end surface 72, the second end surface 72 is in communication with an end of the air inlet channel 232, the air guide member 70 has an air guide channel 73, and two opposite ends of the air guide channel 73 are respectively in communication with the first end surface 71 and the second end surface 72.

In an embodiment, the air guide member 70 further has a second mounting hole 74, and the second mounting hole 74 is aligned with the first mounting hole 52. When the airflow channel 61 is formed by the vent tube 60, the vent tube 60 is assembled in the first mounting hole 52 and an end thereof extends into the second mounting hole 74, so as to fix the vent tube 60 while achieving air guide. Air entering the air inlet channel 232 enters the second mounting hole 74 through a side of the second end surface 72, and then enters the airflow channel 61 inside the vent tube 60.

In an embodiment, referring to FIG. 2, the vaporizer 100 further includes a positive connecting member 90, and an end of the heating base 23 in the axial direction L further has a negative connecting member 231. The positive connecting member 90 extends through the air guide member 70 to be connected to a positive electrode of the heating element, and the negative connecting member 231 is connected to a negative electrode of the heating element. The positive connecting member 90 and the negative connecting member 231 are both connected to electrical elements through the heating base 23 to provide electrical energy for the heating of the heating element.

Further, the negative connecting member 231 may be formed as an external thread shape for detachable connection to the electrical elements.

In the vaporizer 100 provided in this application, the end of the airflow channel 61 in communication with the vaporization surface 32 is close to the heating element. During heating of the vaporization surface 32 by the heating element, the heat of the heating element is transmitted to the aerosol-forming substrate near the liquid inlet end 51a of the liquid guide channel 51 through the airflow channel 61, to reduce the viscosity of the aerosol-forming substrate, so as to reduce the resistance to air replenishment from the airflow channel 61 to the liquid inlet end 51a, thereby facilitating successful entry of a venting airflow in the airflow channel 61 to the liquid inlet end 51a for air replenishment. Moreover, while the airflow is rapidly replenished through the airflow channel 61, the airflow continuously flows in the airflow channel 61. The presence of the airflow can prevent reverse leakage of liquid through the airflow channel 61, thereby ensuring smooth ventilation while avoiding reverse leakage.

An embodiment of the utility model further provides an electronic vaporization device. The electronic vaporization device includes the vaporizer 100 provided in any of the above embodiments. Since the electronic vaporization device has all technical features of the vaporizer 100 provided in any of the above embodiments, the electronic vaporization device has the same technical effects as the above vaporizer 100.

The technical features in the foregoing embodiments may be randomly combined. For concise description, not all possible combinations of the technical features in the embodiments are described. However, provided that combinations of the technical features do not conflict with each other, the combinations of the technical features are considered as falling within the scope described in the description.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A vaporizer, comprising:
a tube body having a liquid guide channel therein;
a liquid guide member having a liquid guide surface and a vaporization surface arranged opposite each other, the liquid guide surface being in communication with a liquid inlet end of the liquid guide channel and configured to guide an aerosol-forming substrate from the liquid guide surface to the vaporization surface; and
a heating element arranged on the vaporization surface and configured to heat and vaporize the aerosol-forming substrate,
wherein the tube body comprises an air inlet channel,
wherein the vaporization surface is in communication with the air inlet channel,
wherein an airflow channel is constructed inside the tube body, and
wherein two opposite ends of the airflow channel are respectively in communication with the liquid inlet end of the liquid guide channel and the vaporization surface.

2. The vaporizer of claim 1, further comprising:
a vent tube arranged in the tube body,
wherein the airflow channel is formed inside the vent tube.

3. The vaporizer of claim 1, wherein, in a direction from the vaporization surface to the liquid guide surface, an end of the airflow channel is flush with the liquid inlet end of the liquid guide channel or an end of the airflow channel is higher than the liquid inlet end of the liquid guide channel.

4. The vaporizer of claim 1, further comprising:
a first seal member arranged between the tube body and the liquid guide member,
wherein the liquid guide channel and the airflow channel are both arranged on the first seal member and extend in an axial direction of the tube body.

5. The vaporizer of claim 4, further comprising:
an air guide member arranged in the tube body and located on a side of the liquid guide member facing away from the first seal member,
wherein the air guide member, the liquid guide member, and the tube body jointly define a vaporization cavity,
wherein the vaporization surface is arranged facing the vaporization cavity, and
wherein the air inlet channel is in communication with the vaporization cavity.

6. The vaporizer of claim 5, wherein the air guide member has a first end surface,
wherein a vaporization groove with an opening is arranged on the first end surface, and
wherein the first end surface is attached to the first seal member for the liquid guide member to cover an opening of the vaporization groove and define the vaporization cavity.

7. The vaporizer of claim 5, wherein the air guide member has a first end surface spaced apart from the liquid guide member in the axial direction of the tube body, and
wherein the first end surface, the tube body, and the liquid guide member define the vaporization cavity.

8. The vaporizer of claim 6, wherein the air guide member has a second end surface in communication with an end of the air inlet channel, and
wherein the air guide member has an air guide channel, and
wherein two opposite ends of the air guide channel are respectively in communication with the first end surface and the second end surface.

9. The vaporizer of claim 1, further comprising:
a housing sleeved on an outer periphery of the tube body and defining a liquid storage cavity with the tube body,
wherein the tube body has a liquid inlet hole facing the liquid storage cavity, the liquid inlet hole forming the liquid inlet end of the liquid guide channel, and
wherein the liquid storage cavity is in communication with the liquid guide surface through the liquid inlet hole.

10. The vaporizer of claim 2, wherein the vent tube comprises at least one capillary tube, and
wherein a pore size of the capillary tube is in a range of 0.15 mm to 0.45 mm.

11. An electronic vaporization device, comprising:
the vaporizer of claim 1.
